(19) United States
(12) Patent Application Publication
(10) Pub. No.: US 2022/0089642 A1
(43) Pub. Date: Mar. 24, 2022

(54) Fc Receptor-ACE2 Conjugates and Use Thereof

(71) Applicant: CITY OF HOPE, Duarte, CA (US)

(72) Inventors: John C. WILLIAMS, Monrovia, CA (US); Miso PARK, Duarte, CA (US)

(21) Appl. No.: 17/478,824

(22) Filed: Sep. 17, 2021

Related U.S. Application Data
(60) Provisional application No. 63/079,970, filed on Sep. 17, 2020.

(51) Int. Cl. C07K 7/08; C12N 15/86; C07K 16/08

(52) U.S. Cl. CPC C07K 7/08; A61K 38/162; C07K 16/08; C12N 15/86

(57) ABSTRACT

Provided herein are, inter alia, peptides capable of binding viral proteins and thereby preventing viral infection, replication and spread (e.g., SARS CoV-2). The conjugates provided herein include an dimerizing domain (e.g., Fc domain) attached through a peptide linker to a protein domain (viral protein binding domain). The viral protein binding domain is capable of binding a viral protein, for example, a viral envelope protein or a portion thereof.

### CROSS REFERENCE TO RELATED APPLICATIONS

This application claims priority to U.S. Provisional Application No. 63/079,970, filed Sep. 17, 2020, which is hereby incorporated by reference in its entirety and for all purposes.

### REFERENCE TO A SEQUENCE LISTING, A TABLE OR A COMPUTER PROGRAM LISTING APPENDIX SUBMITTED AS AN ASCII TEXT FILE

The Sequence Listing written in file 048440-782001US_SL_ST25.txt, created on Sep. 17, 2021, 34,735 bytes, machine format IBM-PC, MS Windows operating system, is hereby incorporated by reference.

### BACKGROUND

SARS-CoV-2 has caused an unprecedented problem, resulting in unaccountable economic and social loss. Previous epidemics and pandemics included those caused by the Spanish Flu Virus, Avian Flu Virus, SARS (or SAARS-CoV-1), Middle East Respiratory Syndrome (MERS), Zika Virus, and Ebola Virus. There is a need in the art for methods and compositions helping to prevent or stop the viral spread. The methods and compositions provided herein, inter alia, address this need and solve other problems in the ar.

### BRIEF SUMMARY

In an aspect is provided a peptide including a dimerizing domain bound to a viral protein binding domain through a chemical linker, wherein the viral protein binding domain is bound to the C-terminus of the dimerizing domain.

In an aspect is provided a peptide complex including: (i) a first peptide including a first dimerizing domain bound to a first viral protein binding domain through a first chemical linker, wherein the first viral protein binding domain is bound to the C-terminus of the first dimerizing domain; and (ii) a second peptide including a second dimerizing domain bound to a second viral protein binding domain through a second chemical linker, wherein the second viral protein binding domain is bound to the C-terminus of the second dimerizing domain; wherein the first dimerizing domain and the second dimerizing domain are bound together thereby binding the first peptide to the second peptide.

In an aspect is provided an isolated nucleic acid encoding a peptide as disclosed herein, including embodiments thereof.

In an aspect is provided an expression vector including a nucleic acid as disclosed herein, including embodiments thereof.

In an aspect is provided a method of treating a viral disease in a subject in need thereof, the method including administering to a subject a therapeutically effective amount of a peptide as disclosed herein including embodiment thereof, thereby treating an infectious disease in the subject.

In an aspect is provided a pharmaceutical composition including a therapeutically effective amount of a peptide as disclosed herein including embodiments thereof and a pharmaceutically acceptable excipient.

### BRIEF DESCRIPTION OF THE DRAWINGS

FIG. 1 shows three-dimensional representations of a conventional approach used to fuse the C-terminus of an ACE2 protein to the N-terminus of an antibody (here a crystallisable fragment (Fc) of an antibody), requiring the use of very long linkers. In this figure, distances are shown in Angstrom (Å). The top view of the fused construct is shown in this figure and the different domains and linkers are labeled shown with arrows.

FIG. 2 shows three-dimensional representations of fusing the N-terminus of an ACE2 protein to the C-terminus of an antibody (here a crystallisable fragment (Fc) of an antibody), requiring the use of short linkers, thereby forming the conjugate provided herein including embodiments thereof. In this figure, distances are shown in Angstrom (A). The top and side views of the fused construct is shown in this figure and the different domains and linkers are labeled shown with arrows.

FIG. 3 shows two schematic representations of a conventional approach for fusing the C-terminus of an ACE2 protein to the N-terminus of an antibody (here a crystallisable fragment (Fc) of an antibody), requiring the use of very long linkers.

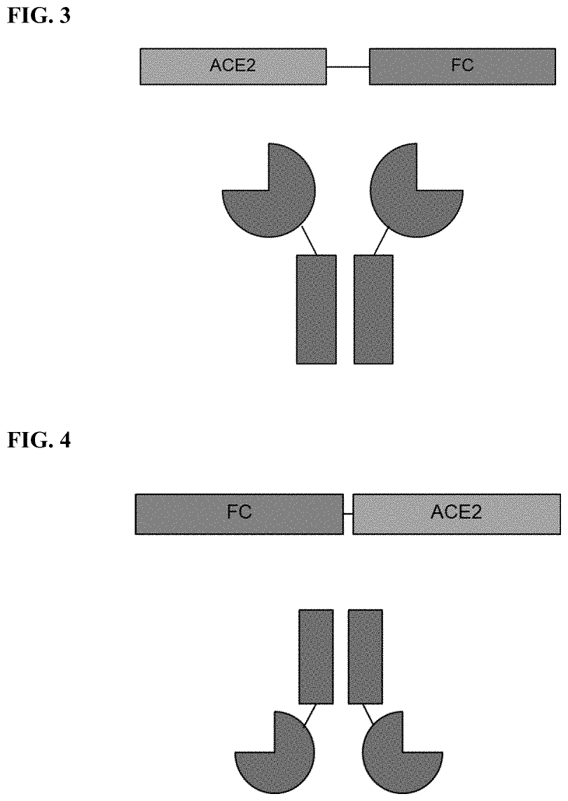

FIG. 4 shows two schematic representations of a novel approach for fusing the N-terminus of an ACE2 protein to the C-terminus of an antibody (here a crystallisable fragment (Fc) of an antibody), requiring the use of short linkers, thereby forming the conjugate provided herein including embodiments thereof.

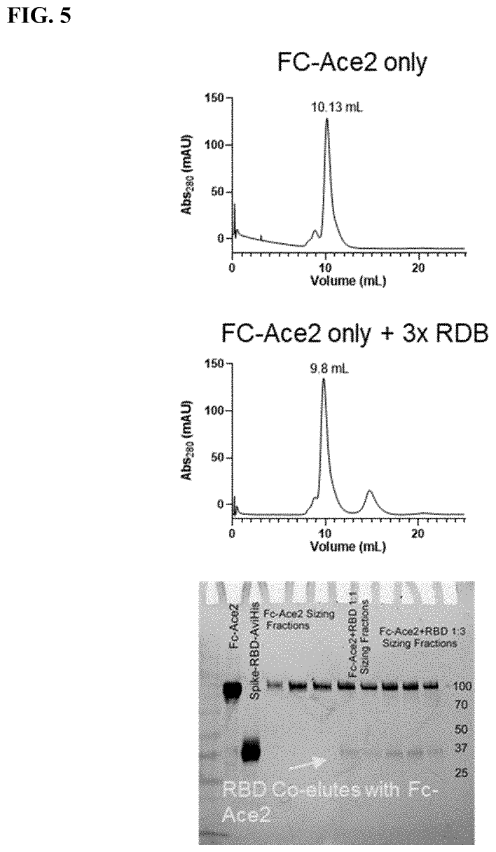

FIG. 5 presents two line graphs of elution profiles and a picture of a Western-blot showing that the Fc-ACE2 construct binds to the receptor binding domain (RBD) of the spike protein.

FIG. 6 is a line graph showing that the Fc-ACE2 construct binds to the trimeric Spike protein. In this experiment, the trimeric spike protein was tethered to a surface plasmon resonance (SPR) chip, and different concentrations of the Fc-ACE2 were passed over the SPR chip. The calculated KD was beyond the instrument limit.

FIG. 7 is a line graph showing that the Fc-ACE2 construct block viral entry in a pseudovirus assay comparing soluble ACE2, the ACE2-Fc construct, and the FC-ACE2 construct. In these experiments, the linker used in the ACE2-Fc construct was 27 residues long. The linker used in the Fc-ACE2 construct was 10 residues long.

FIG. 8A-8D Results of pseudovirus neutralization assays are shown. The assays were done with plates seeded with 1.25E10+4 HEK293T-ACE2 cells in 50 uL DMEM with 10% heat-inactivated FBS, 2 mM L-glutamine. 1:81 Wuhan-Hu-1 Spike-Pseudotyped Lentivirus was added 1:1 with each treatment, incubated for 1 hour at 37 C, 5% CO2, added to cells and incubated for 48 hours. The plates were read with Bright-Glo Luciferase Assay System. FIG. 8A: IC50: 0.6171 R2: 0.89; FIG. 8B: IC50: 0.7267 R2: 0.92; FIG. 8C: IC50: 0.3754 R2: 0.89. FIG. 8D is a line graph showing that the Fc-ACE2 construct binds to the trimeric Spike protein. In this experiment, the spike hexaprotein was tethered to a surface plasmon resonance (SPR) chip, and different concentrations of the Fc-ACE2 were passed over the SPR chip. 500 RU Spike Hexaprotein Ka: 1.83E5 (1/Ms) Kd: 2.74E-4 (1/s) KD: 4.3 nM

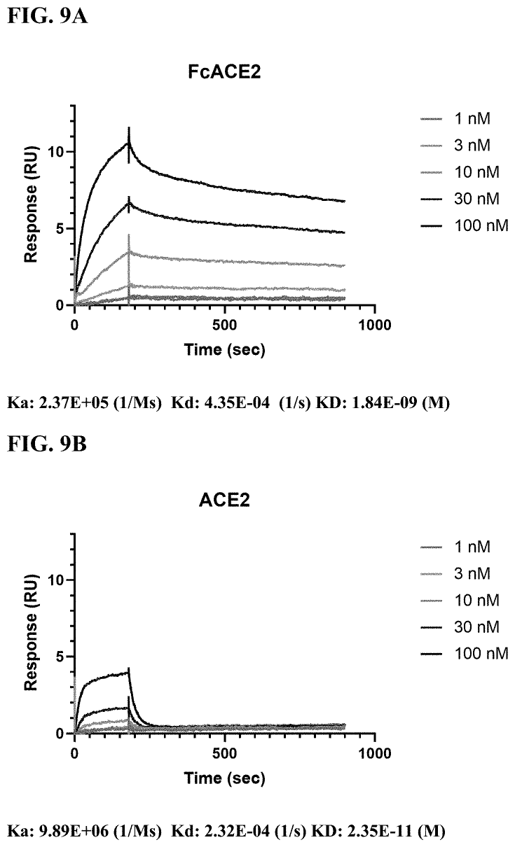

FIG. 9A-9C: Surface Plasmon Resonance (SPR) of FcACE2 peptides provided herein including embodiments thereof with SARS CoV1. CM5 chip immobilized with SARS-CoV1 at 100 RU.

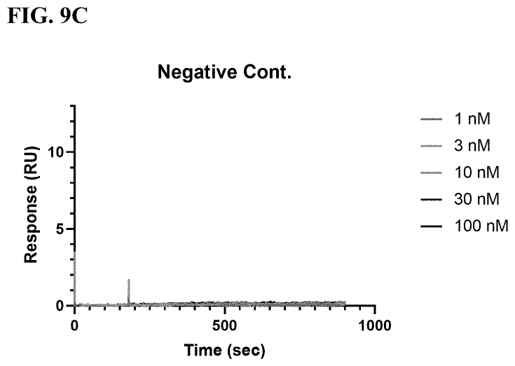

### DETAILED DESCRIPTION

### Definitions

As used herein, the singular forms “a”, “an” and “the” are intended to include the plural forms as well, unless the context clearly indicates otherwise.

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. For example, a selected residue in a selected antibody (or Fab domain) corresponds to light chain threonine at Kabat position 40, when the selected residue occupies the same essential spatial or other structural relationship as a light chain threonine at Kabat position 40. In some embodiments, where a selected protein is aligned for maximum homology with the light chain of an antibody (or Fab domain), the position in the aligned selected protein aligning with threonine 40 is said to correspond to threonine 40. Instead of a primary sequence alignment, a three dimensional structural alignment can also be used, e.g., where the structure of the selected protein is aligned for maximum correspondence with the light chain threonine at Kabat position 40, and the overall structures compared. In this case, an amino acid that occupies the same essential position as threonine 40 in the structural model is said to correspond to the threonine 40 residue.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The terms “corresponding to” and “at a position equivalent to” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue or is “at a position equivalent to” another position when it occupies the same essential structural position within the protein as the given residue. For example, precise amino acid numbering assignments my change between homologous proteins or between versions of the same proteins that differ in length (e.g. due to elimination of a protein domain). Thus, an amino acid residue “at a position equivalent to” another position may be the precise same amino acid position within the context of a given protein domain, but its number assignment may differ due to length between two version of the same protein. An amino acid or nucleotide base “position” is denoted by a number that sequentially identifies each amino acid (or nucleotide base) in the reference sequence based on its position relative to the N-terminus (or 5′-end). As descried above, due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence. By aligning sequences using methods known in the art, a given amino acid position that “corresponds to” or is “equivalent to” a given numbers position is easily identified.

“Nucleic acid” refers to nucleotides (e.g., deoxyribonucleotides or ribonucleotides) and polymers thereof in either single-, double- or multiple-stranded form, or complements thereof, or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab₂, bispecific Fab₂, trispecific Fab₃, monovalent IgGs, scFv, bispecific antibodies, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc (crystallisable fragment) domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable heavy chain provided herein includes in N-terminal to C-terminal direction a CDR H1, a CDR H2 and a CDR H3.

The terms “FR L1”, “FR L2”, “FR L3” and “FR L4” as provided herein are used according to their common meaning in the art and refer to the framework regions (FR) 1, 2, 3 and 4 of the variable light (L) chain of an antibody. In embodiments, the variable light chain provided herein includes in N-terminal to C-terminal direction a FR L1, a FR L2, a FR L3 and a FR L4. Likewise, the terms “FR H1”, “FR H2”, “FR H3” and “FR H4” as provided herein are used according to their common meaning in the art and refer to the framework regions (FR) 1, 2, 3 and 4 of the variable heavy (H) chain of an antibody. In embodiments, the variable heavy chain provided herein includes in N-terminal to C-terminal direction a FR H1, a FR H2, a FR H3 and a FR H4.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

The terms “KD”, “Kd”, “K_{D}” or “K_{d}” are used according to its commonly known meaning in the art. A dissociation constant is a specific type of equilibrium constant that measures the propensity of a larger object to separate (dissociate) reversibly into smaller components, as when a complex falls apart into its component molecules, or when a salt splits up into its component ions. The dissociation constant is the inverse of the association constant. KD is the equilibrium dissociation constant, a ratio of k_{off}/kₒₙ, between the antibody and its antigen. KD and affinity are inversely related. The KD value relates to the concentration of antibody (the amount of antibody needed for a particular experiment) and so the lower the KD value (lower concentration) and thus the higher the affinity of the antibody.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). The term “antibody” as referred to herein further includes antibody variants such as single domain antibodies. Thus, in embodiments an antibody includes a single monomeric variable antibody domain. Thus, in embodiments, the antibody, includes a variable light chain (VL) domain or a variable heavy chain (VH) domain. In embodiments, the antibody is a variable light chain (VL) domain or a variable heavy chain (VH) domain. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “spike protein”, “S protein”, or “SARS-CoV-2 S protein” are used in accordance with their plain meaning as understood in the art and refer to the spike (S) protein of the SARS-CoV-2, or variants or homologs thereof. TIn embodiments, the S protein is a large (approx. 180 kDa) glycoprotein. In embodiments, the S protein is present on the viral surface as a trimer. The S protein may include two domains, S1 and S2. In embodiments, the S1 domain mediates receptor binding and is divided into two sub-domains, with the N-terminal subdomain (NTD) often binding sialic acid and the C-terminal subdomain (also known as C-domain) binding a specific proteinaceous receptor. In embodiments, the S2 domain mediates viral-membrane fusion through the exposure of a highly conserved fusion peptide. The fusion peptide may be activated through proteolytic cleavage at a site found immediately upstream (S2′), which is common to all coronaviruses. In many (but not all) coronaviruses, additional proteolytic priming may occur at a second site located at the interface of the S1 and S2 domains (S1/S2). In some embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring the S protein polypeptide (e.g. YP_009724390.1). In embodiments, the S protein is the protein as identified by the NCBI sequence reference YP_009724390.1, homolog or functional fragment thereof.

The term “ACE2” or “angiotensin converting enzyme 2” as referred to herein are used in accordance with their plain meaning as understood in the art and refer to any of the recombinant or naturally-occurring forms of the ACE2 enzyme, or variants or homologs thereof that maintain ACE2 enzyme activity (e.g. within at least 50%, 80%, 90%, 95%, 96%, 97%, 98%, 99% or 100% activity compared to ACE2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ACE2 protein. In embodiments, the ACE2 protein is substantially identical to the protein identified by the UniProt reference number Q9BYF1 or a variant or homolog having substantial identity thereto. ACE2 is a zinc containing metalloenzyme which catalyzes the conversion of angiotensin II (Ang 1-8) to angiotensin (Ang 1-7), which is a vasodilator. ACE2 also is the cellular receptor for sudden acute respiratory syndrome (SARS) coronavirus/SARS-CoV and human coronavirus NL63/HCoV-NL63.

The terms “virus” or “virus particle” are used according to its plain ordinary meaning within Virology and refers to a virion including the viral genome (e.g. DNA, RNA, single strand, double strand), viral capsid and associated proteins, and in the case of enveloped viruses (e.g. herpesvirus), an envelope including lipids and optionally components of host cell membranes, and/or viral proteins.

The term “replicate” is used in accordance with its plain ordinary meaning and refers to the ability of a cell or virus to produce progeny. A person of ordinary skill in the art will immediately understand that the term replicate when used in connection with DNA, refers to the biological process of producing two identical replicas of DNA from one original DNA molecule. In the context of a virus, the term “replicate” includes the ability of a virus to replicate (duplicate the viral genome and packaging said genome into viral particles) in a host cell and subsequently release progeny viruses from the host cell, which results in the lysis of the host cell. A “replication-competent” virus as provided herein refers to a virus (chimeric poxvirus) that is capable of replicating in a cell (e.g., a cancer cell). Similarly, an “oncolytic virus” as referred to herein, is a virus that is capable of infecting and killing cancer cells. As the infected cancer cells are destroyed by oncolysis, they release new infectious virus particles or virions to help destroy the remaining tumor. In embodiments, the chimeric poxvirus is able to replicate in a cancer cell. In embodiments, the chimeric poxvirus does not detectably replicate in a healthy cell relative to a standard control. In embodiments, the chimeric poxvirus provided herein has an increased oncolytic activity compared to its parental virus. In embodiments, the oncolytic activity (ability to induce cell death in an infected cell) is more than 1.5, 2, 3, 4, 5, 6, 7, 8, 9, 10, 100, 10000, 10000 times increased compared to the oncolytic activity of a parental virus (one of the viruses used to form the chimeric virus provided herein).

The terms “SARS-CoV-2” or “SARS-CoV 2” refer to the Severe Acute Respiratory Syndrome Coronavirus 2. In embodiments, the SARS-CoV-2 is the strain of coronavirus that causes coronavirus disease 2019 (COVID-19), the respiratory illness responsible for the COVID-19 pandemic. In embodiments, the SARS-CoV-2 is colloquially known as simply the coronavirus, it was previously referred to by its provisional name, 2019 novel coronavirus (2019-nCoV), and has also been called human coronavirus 2019 (HCoV-19 or hCoV-19). In embodiments, the SARS-CoV-2 is a Baltimore class IV positive-sense single-stranded RNA virus that is contagious in humans.

The terms “SARS-CoV-1” or “SARS-CoV 1” refer to the Severe Acute Respiratory Syndrome Coronavirus 1. In embodiments, the SARS-CoV-1 is the strain of coronavirus that causes the respiratory illness responsible for the 2002-2004 SARS outbreak. In embodiments, the SARS-CoV-1 is colloquially known as the virus causing SARS. In embodiments, the SARS-CoV-1 is an enveloped, positive-sense, single-stranded RNA virus which infects the epithelial cells within the lungs. The virus enters the host cell by binding to angiotensin-converting enzyme 2 and infects humans, bats, and palm civets.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “effective dose” or “effective dosage” is defined as an amount sufficient to achieve or at least partially achieve the desired effect.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The term “immune response” used herein encompasses, but is not limited to, an “adaptive immune response”, also known as an “acquired immune response” in which adaptive immunity elicits immunological memory after an initial response to a specific pathogen or a specific type of cells that is targeted by the immune response, and leads to an enhanced response to that target on subsequent encounters. The induction of immunological memory can provide the basis of vaccination.

The term “immunogenic” or “antigenic” refers to a compound or composition that induces an immune response, e.g., cytotoxic T lymphocyte (CTL) response, a B cell response (for example, production of antibodies that specifically bind the epitope), an NK cell response or any combinations thereof, when administered to an immunocompetent subject. Thus, an immunogenic or antigenic composition is a composition capable of eliciting an immune response in an immunocompetent subject. For example, an immunogenic or antigenic composition can include one or more immunogenic epitopes associated with a pathogen or a specific type of cells that is targeted by the immune response. In addition, an immunogenic composition can include isolated nucleic acid constructs (such as DNA or RNA) that encode one or more immunogenic epitopes of the antigenic polypeptide that can be used to express the epitope(s) (and thus be used to elicit an immune response against this polypeptide or a related polypeptide associated with the targeted pathogen or type of cells).

The term “EC50” or “half maximal effective concentration” as used herein refers to the concentration of a molecule (e.g., antibody, chimeric antigen receptor or bispecific antibody) capable of inducing a response which is halfway between the baseline response and the maximum response after a specified exposure time. In embodiments, the EC50 is the concentration of a molecule (e.g., antibody, chimeric antigen receptor or bispecific antibody) that produces 50% of the maximal possible effect of that molecule.

An “inhibitor” refers to a compound (e.g. compounds described herein) that reduces activity when compared to a control, such as absence of the compound or a compound with known inactivity.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “associated” or “associated with” in the context of a substance or substance activity or function associated with a disease (e.g. a protein associated disease, a cancer (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, synoviocytes, synovial fluid, synovial tissue, fibroblast-like synoviocytes, macrophagelike synoviocytes, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “COVID19”, “COVID-19” refer to the coronavirus disease 2019, caused by SARS-CoV-2. In embodiments, the COVID-19 is a respiratory illness characterized by symptoms such as fever, cough, loss of appetite, fatigue, shortness of breath, coughing up sputum, muscle aches and pains, nausea, vomiting, diarrhea, sneezing, runny nose, sore throat, skin lesions, chest tightness, palpitations, decrease sense or loss of sense of smell, and/or disturbances in sense of taste. Comorbidities of COVID-19 include moderate or severe asthma, pre-existing chronic obstructive pulmonary disease, pulmonary fibrosis, cystic fibrosis, hypertension, diabetes mellitus, and cardiovascular diseases such as, but limited to, coronary artery diseases, stroke, heart failure, hypertensive heart disease, rheumatic heart disease, or cardiomyopathy.

The terms “disease” or “condition” refer to a state of being or health status of a patient or subject capable of being treated with the compounds or methods provided herein.

The term “prophylactic treatment” as used herein, refers to any intervention using the compositions embodied herein, that is administered to an individual in need thereof or having an increased risk of acquiring a respiratory tract infection, wherein the intervention is carried out prior to the onset of a viral infection, e.g. SARS-CoV-2, and typically has in effect that either no viral infection occurs or no clinically relevant symptoms of a viral infection occur in a healthy individual upon subsequent exposure to an amount of infectious viral agent that would otherwise, i.e. in the absence of such a prophylactic treatment, be sufficient to cause a viral infection.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “therapeutically effective dose or amount” as used herein is meant a dose that produces effects for which it is administered (e.g. treating or preventing a disease such as COVID-19 and its implications). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

A “ligand” refers to an agent, e.g., a polypeptide or other molecule, capable of binding to a receptor or antibody, antibody variant, antibody region or fragment thereof.

The term “therapy” or “therapeutic treatment” as used herein relates to the administration of the compositions embodied herein, in order to achieve a reduction in severity and/or frequency of symptoms, elimination of symptoms and/or underlying cause, prevention of the occurrence of symptoms and/or their underlying cause, and/or improvement or remediation of damage directly caused by or indirectly associated, e.g. through secondary infection, with the viral infection.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

“Treatment”, or “treating” as used herein, is defined as the application or administration of a therapeutic agent or combination of therapeutic agents to a patient, or application or administration of the active agent to a patient, who has a virus infection, e.g. SARS-CoV-2, with the purpose to cure, heal, alleviate, relieve, alter, remedy, ameliorate, improve or affect the infection, or symptoms thereof. The term “treatment” or “treating” is also used herein in the context of administering agents prophylactically. Accordingly, “treating” or “treatment” of a state, disorder or condition includes: (1) eradicating the virus; (2) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (3) inhibiting the state, disorder or condition, i.e., arresting, reducing or delaying the development of the disease or a relapse thereof (in case of maintenance treatment) or at least one clinical or subclinical symptom thereof, or (4) relieving the disease, i.e., causing regression of the state, disorder or condition or at least one of its clinical or subclinical symptoms. The benefit to an individual to be treated is either statistically significant or at least perceptible to the patient or to the physician.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and. This applies regardless of the breadth of the range.

Any compositions or methods provided herein can be combined with one or more of any of the other compositions and methods provided herein.

A number of embodiments have been described. Nevertheless, it will be understood that various modifications may be made. Accordingly, other embodiments are within the scope of the claims.

### Recombinant Peptides

Provided herein are, inter alia, peptides capable of binding viral proteins and thereby preventing viral infection, replication and spread (e.g., SARS CoV-2, SARS CoV-1). The conjugates provided herein include an dimerizing domain (e.g., Fc domain) attached through a peptide linker to a protein domain (viral protein binding domain). The viral protein binding domain is capable of binding a viral protein, for example, a viral envelope protein or a portion thereof. In embodiments, the viral protein binding domain is a domain of a cellular protein (e.g., ACE2) or a variant thereof. Through the peptide linker, the viral protein binding domain is attached to the C-terminus of the dimerizing domain (e.g., Fc domain). In contrast to previously made conjugates where the viral protein binding domain is attached to the N-terminus of an Fc domain, the peptides provided herein superior affinities enhance through avidity.

In an aspect is provided a peptide including a dimerizing domain bound to a viral protein binding domain through a chemical linker, wherein the viral protein binding domain is bound to the C-terminus of the dimerizing domain.

A “viral protein binding domain” as provided herein is a protein domain capable of binding a viral protein or fragment thereof. In embodiments, the viral protein binding domain is a Severe Acute Respiratory Syndrome (SARS)-coronavirus (CoV) protein binding domain or a Middle East Respiratory Syndrome (MERS)-coronavirus (CoV) protein binding domain. In embodiments, the viral protein binding domain is a Severe Acute Respiratory Syndrome (SARS)-coronavirus (CoV) protein binding domain. In embodiments, the viral protein binding domain is a Middle East Respiratory Syndrome (MERS)-coronavirus (CoV) protein binding domain. In embodiments, the viral protein binding domain is a Severe Acute Respiratory Syndrome (SARS)-coronavirus (CoV)-1 protein binding domain. In embodiments, the viral protein binding domain is a Severe Acute Respiratory Syndrome (SARS)-coronavirus (CoV)-2 protein binding domain.

In embodiments, the viral protein binding domain is a viral envelop protein binding domain. In embodiments, the viral protein binding domain is a spike protein binding domain. In embodiments, the viral protein binding domain is a SARS CoV-2 protein binding domain. In embodiments, the viral protein binding domain is a SARS CoV-1 protein binding domain. In embodiments, the viral protein binding domain is a SARS CoV-2 receptor binding domain (RBD) binding domain. In embodiments, the viral protein binding domain is a cellular protein or a fragment thereof. In embodiments, the viral protein binding domain is an ACE2 domain.

In embodiments, the viral protein binding domain includes the sequence of SEQ ID NO:1. In embodiments, the viral protein binding domain is the sequence of SEQ ID NO:1. In embodiments, the viral protein binding domain includes the sequence of SEQ ID NO:6. In embodiments, the viral protein binding domain is the sequence of SEQ ID NO:6. In embodiments, the viral protein binding domain includes the sequence of SEQ ID NO: 7. In embodiments, the viral protein binding domain is the sequence of SEQ ID NO:7. In embodiments, the viral protein binding domain includes the sequence of SEQ ID NO: 8. In embodiments, the viral protein binding domain is the sequence of SEQ ID NO: 8.

In embodiments, the angiotensin converting enzyme 2 (ACE2) domain is an enzymatically active ACE domain. In embodiments, the ACE2 domain is an enzymatically inactive ACE domain. An “enzymatically inactive ACE2 domain” as provided herein does not have detectable enzymatic activity relative to a standard control (e.g., an enzymatically active ACE protein).

In embodiments, the peptide has an IC50 (half-maximal inhibitory concentration) of about 0.2 to about 0.9. In embodiments, the peptide has an IC50 of about 0.3 to about 0.9. In embodiments, the peptide has an IC50 of about 0.4 to about 0.9. In embodiments, the peptide has an IC50 of about 0.5 to about 0.9. In embodiments, the peptide has an IC50 of about 0.6 to about 0.9. In embodiments, the peptide has an IC50 of about 0.7 to about 0.9. In embodiments, the peptide has an IC50 of about 0.8 to about 0.9. In embodiments, the peptide has an IC50 of about 0.6171. In embodiments, the peptide has an IC50 of about 0.7267. In embodiments, the peptide has an IC50 of about 0.3754.

In embodiments, the peptide has an IC50 of 0.2 to 0.9. In embodiments, the peptide has an IC50 of 0.3 to 0.9. In embodiments, the peptide has an IC50 of 0.4 to 0.9. In embodiments, the peptide has an IC50 of 0.5 to 0.9. In embodiments, the peptide has an IC50 of 0.6 to 0.9. In embodiments, the peptide has an IC50 of 0.7 to 0.9. In embodiments, the peptide has an IC50 of 0.8 to 0.9. In embodiments, the peptide has an IC50 of 0.6171. In embodiments, the peptide has an IC50 of 0.7267. In embodiments, the peptide has an IC50 of 0.3754.

The ability of a peptide to bind a second peptide can be described by the equilibrium dissociation constant (K_{D}). The equilibrium dissociation constant (K_{D}) as defined herein is the ratio of the dissociation rate (K-off) and the association rate (K-on) of a peptide to a viral protein. It is described by the following formula: K_{D}=K-off/K-on. In embodiments, the peptide is capable of binding a viral protein with an equilibrium dissociation constant (K_{D}) from about 1 nM to about 10 nM. In embodiments, the peptide is capable of binding a viral protein with an equilibrium dissociation constant (K_{D}) from about 1 nM to about 9 nM. In embodiments, the peptide is capable of binding a viral protein with an equilibrium dissociation constant (K_{D}) from about 1 nM to about 8 nM. In embodiments, the peptide is capable of binding a viral protein with an equilibrium dissociation constant (K_{D}) from about 1 nM to about 7 nM. In embodiments, the peptide is capable of binding a viral protein with an equilibrium dissociation constant (K_{D}) from about 1 nM to about 6 nM. In embodiments, the peptide is capable of binding a viral protein with an equilibrium dissociation constant (K_{D}) from about 1 nM to about 5 nM. In embodiments, the peptide is capable of binding a viral protein with an equilibrium dissociation constant (K_{D}) from about 1 nM to about 4 nM. In embodiments, the peptide is capable of binding a viral protein with an equilibrium dissociation constant (K_{D}) from about 1 nM to about 3 nM. In embodiments, the peptide is capable of binding a viral protein with an equilibrium dissociation constant (K_{D}) from about 1 nM to about 2 nM. In embodiments, the peptide is capable of binding a viral protein with an equilibrium dissociation constant (K_{D}) of about 4.3 nM. In embodiments, the peptide is capable of binding a viral protein with an equilibrium dissociation constant (K_{D}) of about 1.8 nM.

The peptide provided herein including embodiments thereof may be bound to a viral protein through the viral protein binding domain (e.g., ACE2 domain). In embodiments, the peptide binds a viral protein through the viral protein binding domain (e.g., through the first viral protein binding domain or through the second viral protein binding domain, or through the first viral protein binding domain and the second viral protein binding domain). In embodiments, the peptide binds a viral protein through the viral protein binding domain with an equilibrium dissociation constant (K_{D}) from 1 nM to 10 nM. In embodiments, the peptide is capable of binding a viral protein with an equilibrium dissociation constant (K_{D}) from 1 nM to 9 nM. In embodiments, the peptide is capable of binding a viral protein with an equilibrium dissociation constant (K_{D}) from 1 nM to 8 nM. In embodiments, the peptide is capable of binding a viral protein with an equilibrium dissociation constant (K_{D}) from 1 nM to 7 nM. In embodiments, the peptide is capable of binding a viral protein with an equilibrium dissociation constant (K_{D}) from 1 nM to 6 nM. In embodiments, the peptide is capable of binding a viral protein with an equilibrium dissociation constant (K_{D}) from 1 nM to 5 nM. In embodiments, the peptide is capable of binding a viral protein with an equilibrium dissociation constant (K_{D}) from 1 nM to 4 nM. In embodiments, the peptide is capable of binding a viral protein with an equilibrium dissociation constant (K_{D}) from 1 nM to 3 nM. In embodiments, the peptide is capable of binding a viral protein with an equilibrium dissociation constant (K_{D}) from 1 nM to 2 nM. In embodiments, the peptide is capable of binding a viral protein with an equilibrium dissociation constant (K_{D}) of 4.3 nM. In embodiments, the peptide is capable of binding a viral protein with an equilibrium dissociation constant (K_{D}) of 1.8 nM. In embodiments, the peptide is capable of binding a viral protein with an equilibrium dissociation constant (K_{D}) of 1.0, 1.1, 1.2., 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0. 4.1, 4.2, 4.3, 4.4, 4.5, 4.6, 4.7, 4.8. 4.9, 5.0, 5.1, 5.2, 5.3, 5.4, 5.5, 5.6, 5.7, 5.8, 5.9 or 6.0 nM.

In embodiments, the ACE2 domain includes an ACE2 extracellular domain, wherein the ACE2 extracellular domain includes an amino acid mutation that decreases enzymatic activity relative to an ACE2 protein without said amino acid mutation. In embodiments, the ACE2 domain binds to a SARS-CoV-2 Spike protein. In embodiments, the ACE2 domain binds to a SARS-CoV-1 Spike protein. In embodiments, the ACE2 domain binds to a SARS-CoV-2 Spike protein with a Kd of less than or equal to that of an ACE2 protein without the amino acid mutation. In embodiments, the ACE2 domain includes the sequence of SEQ ID NO:6. In embodiments, the ACE2 domain is the sequence of SEQ ID NO:6. In embodiments, the ACE2 domain includes the sequence of SEQ ID NO:7. In embodiments, the ACE2 domain is the sequence of SEQ ID NO:7. In embodiments, the ACE2 domain is the sequence of SEQ ID NO:8. In embodiments, the ACE2 domain includes the sequence of SEQ ID NO:8.

In embodiments, the ACE2 domain of SEQ ID NO:1 includes an amino acid substitution at a position corresponding to position 273 or position 345. In embodiments, the substitution corresponding to position 273 is a R273S substitution. In embodiments, the substitution corresponding to position 345 is a H345F or H345S substitution. In embodiments, the substitution corresponding to position 345 is a H345F substitution. In embodiments, the substitution corresponding to position 345 is a H345S substitution.

In embodiments, the ACE2 domain of SEQ ID NO:1 includes an amino acid substitution at position 273 or at position 345. In embodiments, the substitution at position 273 is a R273S substitution. In embodiments, the substitution at position 345 is a H345F or H345S substitution. In embodiments, the substitution at position 345 is a H345F substitution. In embodiments, the substitution at position 345 is a H345S substitution.

In embodiments, the amino acid substitution is at a position equivalent to position 273. In embodiments, the amino acid substitution is at a position corresponding to position 273. In embodiments, the amino acid substitution at a position corresponding to position 273 is an R to S amino acid substitution.

In embodiments, the amino acid substitution is a R273S substitution. In embodiments, the amino acid substitution is a H345F substitution.

In embodiments, the peptide includes from the N-terminus to the C-terminus a dimerizing domain, a chemical linker, and a viral protein binding domain. In embodiments, the peptide includes the sequence of SEQ ID NO:5. In embodiments, the peptide is the sequence of SEQ ID NO:5.

In embodiments, the dimerizing domain is a Fc dimerizing domain. An “Fc dimerizing domain” as referred to herein is a polypeptide including an antibody CH2 domain or fragment thereof bound (covalently and/or non-covalently) to an antibody CH3 domain or fragment thereof. Upon binding of two dimerizing domains an antibody Fc region is formed. Thus, an Fc region may include a first dimerizing domain non-covalently or covalently bound to a second dimerizing domain. In embodiments, the CH3 domain of the first Fc dimerizing domain is non-covalently bound to the CH3 domain of the second Fc dimerizing domain. In embodiments, the CH2 domain of the first Fc dimerizing domain is covalently bound to the CH2 domain of the second Fc dimerizing domain. In embodiments, the CH2 domain of the first Fc dimerizing domain is bound to the CH2 domain of the second Fc dimerizing domain through a disulfide linkage. In embodiments, the Fc dimerizing domain includes a CH2 domain and a CH3 domain. In embodiments, the Fc dimerizing domain includes from the N-terminus to the C-terminus a CH2 domain and a CH3 domain.

In embodiments, the Fc domain includes a glycine at a position corresponding to amino acid residue 297 of SEQ ID NO:4, a cysteine at a position corresponding to amino acid residue 292 of SEQ ID NO:4, a cysteine at a position corresponding to amino acid residue 302 of SEQ ID NO:4, or a glycine at a position corresponding to amino acid residue 297 of SEQ ID NO:4.

In embodiments, the Fc domain includes a glycine at a position corresponding to amino acid residue 297 of SEQ ID NO:4, a cysteine at a position corresponding to amino acid residue 292 of SEQ ID NO:4, and a cysteine at a position corresponding to amino acid residue 302 of SEQ ID NO:4. In embodiments, the Fc domain includes a glycine at a position corresponding to amino acid residue 297 of SEQ ID NO:4.

In embodiments, the dimerizing domain includes the sequence of SEQ ID NO:2. In embodiments, the dimerizing domain is the sequence of SEQ ID NO:2.

A “chemical linker,” as provided herein, is a covalent linker, a peptide or peptidyl linker (a linker including a peptide moiety), a cleavable peptide linker, a substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene or any combination thereof.

The chemical linker as provided herein may be a bond, —O—, —S—, —C(O)—, —C(O)O—, —C(O)NH—, —S(O)2NH—, —NH—, —NHC(O)NH—, substituted (e.g., substituted with a substituent group, a size-limited substituent or a lower substituent group) or unsubstituted alkylene, substituted (e.g., substituted with a substituent group, a size-limited substituent or a lower substituent group) or unsubstituted heteroalkylene, substituted (e.g., substituted with a substituent group, a size-limited substituent or a lower substituent group) or unsubstituted cycloalkylene, substituted (e.g., substituted with a substituent group, a size-limited substituent or a lower substituent group) or unsubstituted heterocycloalkylene, substituted (e.g., substituted with a substituent group, a size-limited substituent or a lower substituent group) or unsubstituted arylene or substituted (e.g., substituted with a substituent group, a size-limited substituent or a lower substituent group) or unsubstituted heteroarylene.

The chemical linker as provided herein may be a bond, —O—, —S—, —C(O)—, —C(O)O—, C(O)NH—, —S(O)2NH—, —NH—, —NHC(O)NH—, substituted or unsubstituted (e.g., C₁-C₂₀, C₁-C₁₀, C₁-C₅) alkylene, substituted or unsubstituted (e.g., 2 to 20 membered, 2 to 10 membered, 2 to 5 membered) heteroalkylene, substituted or unsubstituted (e.g., C₃-C₈, C₃-C₆, C₃-C₅) cycloalkylene, substituted or unsubstituted (e.g., 3 to 8 membered, 3 to 6 membered, 3 to 5 membered) heterocycloalkylene, substituted or unsubstituted (e.g., C₆-C₁₀, C₆-C₈, C₆-C₅) arylene or substituted or unsubstituted (e.g., 5 to 10 membered, 5 to 8 membered, 5 to 6 membered) heteroarylene.

In embodiments, the chemical linker is a covalent linker. In embodiments, the chemical linker is a hydrocarbon linker. In embodiments, the chemical linker is a peptide linker.

In embodiments, the peptide linker has a length from about 1 to about 15 amino acid residues. In embodiments, the peptide linker has a length from 1 to 15 amino acid residues. In embodiments, the peptide linker has a length of 1, 2, 3, 4, 5, 6, 7, 8, 9, 10, 11, 12, 13, 14 or 15 amino acid residues. In embodiments, the peptide linker has a length of about 1, 2, 3, 4, 5, 6, 7, 8, 9, 10, 11, 12, 13, 14 or 15 amino acid residues. In embodiments, the peptide linker has a length of 1, 2, 3, 4, 5, 6, 7, 8, 9, 10, 11, 12, 13, 14, 15, 16, 17, 18, 19, 20, 21, 22, 23, 24, or 25 amino acid residues. In embodiments, the peptide linker has a length of about 1, 2, 3, 4, 5, 6, 7, 8, 9, 10, 11, 12, 13, 14 15, 16, 17, 18, 19, 20, 21, 22, 23, 24, or 25 amino acid residues.

The chemical linkers provided herein, including embodiments thereof, may have different lengths (e.g., include varying numbers of amino acid residues). Thus, in embodiments, the first chemical linker and the second chemical linker independently have a length of about 1 to about 15 amino acid residues. In embodiments, the first chemical linker and the second chemical linker independently have a length of about 1 to about 14 amino acid residues. In embodiments, the first chemical linker and the second chemical linker independently have a length of about 1 to about 13 amino acid residues. In embodiments, the first chemical linker and the second chemical linker independently have a length of about 1 to about 12 amino acid residues. In embodiments, the first chemical linker and the second chemical linker independently have a length of about 1 to about 11 amino acid residues. In embodiments, the first chemical linker and the second chemical linker independently have a length of about 1 to about 10 amino acid residues. In embodiments, the first chemical linker and the second chemical linker independently have a length of about 1 to about 9 amino acid residues. In embodiments, the first chemical linker and the second chemical linker independently have a length of about 1 to about 8 amino acid residues. In embodiments, the first chemical linker and the second chemical linker independently have a length of about 1 to about 7 amino acid residues. In embodiments, the first chemical linker and the second chemical linker independently have a length of about 1 to about 6 amino acid residues. In embodiments, the first chemical linker and the second chemical linker independently have a length of about 1 to about 5 amino acid residues. In embodiments, the first chemical linker and the second chemical linker independently have a length of about 1 to about 4 amino acid residues. In embodiments, the first chemical linker and the second chemical linker independently have a length of about 1 to about 3 amino acid residues. In embodiments, the first chemical linker and the second chemical linker independently have a length of about 1 to about 2 amino acid residues. In embodiments, the first chemical linker and the second chemical linker independently have a length of about 1 amino acid residues. In embodiments, the first chemical linker and the second chemical linker independently have a length of about 0 amino acid residues.

In embodiments, the first chemical linker and the second chemical linker independently have a length of 1 to 15 amino acid residues. In embodiments, the first chemical linker and the second chemical linker independently have a length of 1 to 14 amino acid residues. In embodiments, the first chemical linker and the second chemical linker independently have a length of 1 to 13 amino acid residues. In embodiments, the first chemical linker and the second chemical linker independently have a length of 1 to 12 amino acid residues. In embodiments, the first chemical linker and the second chemical linker independently have a length of 1 to 11 amino acid residues. In embodiments, the first chemical linker and the second chemical linker independently have a length of 1 to 10 amino acid residues. In embodiments, the first chemical linker and the second chemical linker independently have a length of 1 to 9 amino acid residues. In embodiments, the first chemical linker and the second chemical linker independently have a length of 1 to 8 amino acid residues. In embodiments, the first chemical linker and the second chemical linker independently have a length of 1 to 7 amino acid residues. In embodiments, the first chemical linker and the second chemical linker independently have a length of 1 to 6 amino acid residues. In embodiments, the first chemical linker and the second chemical linker independently have a length of 1 to 5 amino acid residues. In embodiments, the first chemical linker and the second chemical linker independently have a length of 1 to 4 amino acid residues. In embodiments, the first chemical linker and the second chemical linker independently have a length of 1 to 3 amino acid residues. In embodiments, the first chemical linker and the second chemical linker independently have a length of 1 to 2 amino acid residues. In embodiments, the first chemical linker and the second chemical linker independently have a length of 1 amino acid residues. In embodiments, the first chemical linker and the second chemical linker independently have a length of about 0 amino acid residues. In embodiments, the first chemical linker and the second chemical linker independently are a bond.

In embodiments, the peptide linker has a length of less than 20 (e.g., 19, 18, 17, 16, 15, 14, 13, 12, 11, 10, 9, 8, 7, 6, 5, 4, 3, 2, 1, 0) amino acid residues.

In embodiments, the first chemical linker and the second chemical linker independently have a length of 2 to 15 amino acid residues. In embodiments, the first chemical linker and the second chemical linker independently have a length of 3 to 15 amino acid residues. In embodiments, the first chemical linker and the second chemical linker independently have a length of 4 to 15 amino acid residues. In embodiments, the first chemical linker and the second chemical linker independently have a length of 5 to 15 amino acid residues. In embodiments, the first chemical linker and the second chemical linker independently have a length of 6 to 15 amino acid residues. In embodiments, the first chemical linker and the second chemical linker independently have a length of 7 to 15 amino acid residues. In embodiments, the first chemical linker and the second chemical linker independently have a length of 8 to 15 amino acid residues. In embodiments, the first chemical linker and the second chemical linker independently have a length of 9 to 15 amino acid residues. In embodiments, the first chemical linker and the second chemical linker independently have a length of 10 to 15 amino acid residues. In embodiments, the first chemical linker and the second chemical linker independently have a length of 11 to 15 amino acid residues. In embodiments, the first chemical linker and the second chemical linker independently have a length of 12 to 15 amino acid residues. In embodiments, the first chemical linker and the second chemical linker independently have a length of 13 to 15 amino acid residues.

In embodiments, the peptide linker has a length of less than 19 amino acid residues. In embodiments, the peptide linker has a length of less than 18 amino acid residues. In embodiments, the peptide linker has a length of less than 17 amino acid residues. In embodiments, the peptide linker has a length of less than 16 amino acid residues. In embodiments, the peptide linker has a length of less than 15 amino acid residues. In embodiments, the peptide linker has a length of less than 14 amino acid residues. In embodiments, the peptide linker has a length of less than 13 amino acid residues. In embodiments, the peptide linker has a length of less than 12 amino acid residues. In embodiments, the peptide linker has a length of less than 11 amino acid residues. In embodiments, the peptide linker has a length of less than 10 amino acid residues. In embodiments, the peptide linker has a length of less than 9 amino acid residues. In embodiments, the peptide linker has a length of less than 8 amino acid residues. In embodiments, the peptide linker has a length of less than 7 amino acid residues. In embodiments, the peptide linker has a length of less than 6 amino acid residues. In embodiments, the peptide linker has a length of less than 5 amino acid residues. In embodiments, the peptide linker has a length of less than 4 amino acid residues. In embodiments, the peptide linker has a length of less than 3 amino acid residues.

The peptide linker provided herein may be “pasylated.” The term “pasylated” or “pasylation” is used in its customary sense and refers to an amino acid sequence, which due to its high content in proline, alanine and serine forms a highly soluble biological polymer. Thus, in embodiments, the peptide linker includes about 20 proline, alanine and serine residues combined. In embodiments, the peptide linker includes from about 2 to about 20 proline, alanine and serine residues combined. In embodiments, the peptide linker includes hydrophilic residues.

In embodiments, the peptide linker includes the sequence of SEQ ID NO:3. In embodiments, the peptide linker is the sequence of SEQ ID NO:3.

In embodiments, the N-terminus of the viral protein binding domain is bound to a viral protein. In embodiments, the viral protein is a Severe Acute Respiratory Syndrome (SARS)-coronavirus (CoV) protein, or a Middle East Respiratory Syndrome (MERS)-coronavirus (CoV) protein. In embodiments, the viral protein is a Severe Acute Respiratory Syndrome (SARS)-coronavirus (CoV) protein. In embodiments, the viral protein is a Middle East Respiratory Syndrome (MERS)-coronavirus (CoV) protein.

In embodiments, the viral protein is a viral envelop protein. In embodiments, the viral protein is a spike protein. In embodiments, the viral protein is a SARS CoV-2 protein. In embodiments, the viral protein is a SARS CoV-1 protein. In embodiments, the viral protein is a SARS CoV-2 RBD. In embodiments, the viral protein is a SARS CoV-1 RBD.

The peptide provided herein including embodiments thereof may be a first peptide and may form a complex with a second peptide. Through covalent or non-covalent binding of their respective dimerizing domains the first and second peptide may bind to each other thereby forming a peptide complex. Thus, in embodiments, the peptide is a first peptide and the dimerizing domain is a first dimerizing domain.

In embodiments, the first viral protein binding domain is different from the second viral protein binding domain. The first dimerizing domain and the second dimerizing domain may be covalently and/or non-covalently bound to each other. Thus, in embodiments, the first dimerizing domain is bound to the second dimerizing domain. In embodiments, the peptide further includes a covalent bond connecting the first dimerizing domain and the second dimerizing domain.

In embodiments, the first dimerizing domain is bound to a second peptide. The second peptide includes: a second dimerizing domain bound to a second viral protein binding domain through a second chemical linker, wherein the first viral protein binding domain is bound to the C-terminus of the second dimerizing domain; and wherein the first dimerizing domain and the second dimerizing domain are covalently bound together thereby binding the first peptide to the second peptide.

Any of the viral protein binding domains, chemical linkers or dimerizing domains provided herein including embodiments are contemplated for the peptide complexes provided herein. Thus, in embodiments, the first viral protein binding domain and the second viral protein binding domain are each independently an ACE2 domain. In embodiments, the first viral protein binding domain and the second viral protein binding domain each include the sequence of SEQ ID NO:1. In embodiments, the first viral protein binding domain and the second viral protein binding domain each include the sequence of SEQ ID NO:6. In embodiments, the first viral protein binding domain and the second viral protein binding domain each include the sequence of SEQ ID NO:7. In embodiments, the first viral protein binding domain and the second viral protein binding domain each include the sequence of SEQ ID NO:8. In embodiments, the first dimerizing domain and the second protein dimerizing domain are each an Fc dimerizing domain. In embodiments, the first dimerizing domain and the second protein dimerizing domain each include the sequence of SEQ ID NO:2. In embodiments, the first peptide linker and the second peptide linker each include the sequence of SEQ ID NO:3. In embodiments, the first peptide and the second peptide each have the sequence of SEQ ID NO:5.

In embodiments, the second viral protein binding domain is a Severe Acute Respiratory Syndrome (SARS)-coronavirus (CoV) protein binding domain or a Middle East Respiratory Syndrome (MERS)-coronavirus (CoV) protein binding domain. In embodiments, the second viral protein binding domain is a Severe Acute Respiratory Syndrome (SARS)-coronavirus (CoV) protein binding domain. In embodiments, the second viral protein binding domain is a Middle East Respiratory Syndrome (MERS)-coronavirus (CoV) protein binding domain.

In embodiments, the second viral protein binding domain is a viral envelop protein binding domain. In embodiments, the second viral protein binding domain is a spike protein binding domain. In embodiments, the second viral protein binding domain is a SARS CoV-2 protein binding domain. In embodiments, the second viral protein binding domain is a SARS CoV-2 RBD binding domain. In embodiments, the second viral protein binding domain is an ACE2 domain. In embodiments, the second viral protein binding domain includes the sequence of SEQ ID NO:1. In embodiments, the second viral protein binding domain includes the sequence of SEQ ID NO:6. In embodiments, the second viral protein binding domain includes the sequence of SEQ ID NO:7. In embodiments, the second viral protein binding domain includes the sequence of SEQ ID NO:8.

In embodiments, the second dimerizing domain is an Fc dimerizing domain. In embodiments, the second dimerizing domain includes a CH2 and a CH3 domain. In embodiments, the Fc domain includes a glycine at a position corresponding to amino acid residue 297 of SEQ ID NO:4, a cysteine at a position corresponding to amino acid residue 292 of SEQ ID NO:4, a cysteine at a position corresponding to amino acid residue 302 of SEQ ID NO:4, or a glycine at a position corresponding to amino acid residue 297 of SEQ ID NO:4. In embodiments, the Fc domain includes a glycine at a position corresponding to amino acid residue 297 of SEQ ID NO:4, a cysteine at a position corresponding to amino acid residue 292 of SEQ ID NO:4, and a cysteine at a position corresponding to amino acid residue 302 of SEQ ID NO:4. In embodiments, the Fc domain includes a glycine at a position corresponding to amino acid residue 297 of SEQ ID NO:4.

In embodiments, the second dimerizing domain includes the sequence of SEQ ID NO:2. In embodiments, the second chemical linker is a covalent linker. In embodiments, the second chemical linker is a second peptide linker. In embodiments, the second peptide linker has a length from about 1 to about 15 amino acid residues. In embodiments, the second peptide linker has a length of less than 20 amino acid residues. In embodiments, the second peptide linker includes the sequence of SEQ ID NO:3.

In embodiments, the N-terminus of the second viral protein binding domain is bound to a viral protein. In embodiments, the first peptide and the second peptide are chemically different or the same. In embodiments, the second peptide includes from the N-terminus to the C-terminus a second dimerizing domain, a second chemical linker, and a second viral protein binding domain. In embodiments, the second peptide includes the sequence of SEQ ID NO:5. In embodiments, the second peptide is the sequence of SEQ ID NO:5.

In embodiments, the first Fc dimerizing domain includes a first CH2 domain and a first CH3 domain. In embodiments, the second Fc dimerizing domain includes a second CH2 domain and a second CH3 domain. In embodiments, the first CH2 domain is covalently bound to the second CH2 domain. In embodiments, the first CH2 domain is covalently bound to the second CH2 domain through a disulfide linkage. In embodiments, the first CH3 domain is non-covalently bound to the second CH3 domain. In embodiments, the first Fc dimerizing domain and the second Fc dimerizing domain form an antibody Fc region.

In an aspect is provided a peptide complex including: (i) a first peptide including a first dimerizing domain bound to a first viral protein binding domain through a first chemical linker, wherein the first viral protein binding domain is bound to the C-terminus of the first dimerizing domain; and (ii) a second peptide including a second dimerizing domain bound to a second viral protein binding domain through a second chemical linker, wherein the second viral protein binding domain is bound to the C-terminus of the second dimerizing domain; wherein the first dimerizing domain and the second dimerizing domain are bound together thereby binding the first peptide to the second peptide. Any of the viral protein binding domains, chemical linkers or dimerizing domains provided herein including embodiments are contemplated for the peptide complexes provided herein. Thus, in embodiments, the first viral protein binding domain and the second viral protein binding domain each include the sequence of SEQ ID NO:1. In embodiments, the first viral protein binding domain and the second viral protein binding domain each include the sequence of SEQ ID NO:6. In embodiments, the first viral protein binding domain and the second viral protein binding domain each include the sequence of SEQ ID NO:7. In embodiments, the first viral protein binding domain and the second viral protein binding domain each include the sequence of SEQ ID NO:8. In embodiments, the first dimerizing domain and the second protein dimerizing domain each include the sequence of SEQ ID NO:2. In embodiments, the first peptide linker and the second peptide linker each include the sequence of SEQ ID NO:3. In embodiments, the first peptide and the second peptide each have the sequence of SEQ ID NO: 5.

In one embodiment, the peptide includes a dimerizing domain of SEQ ID NO:2, bound to a viral protein binding domain of SEQ ID NO:1 through a chemical linker of SEQ ID NO:3. In one embodiment, the peptide includes a dimerizing domain of SEQ ID NO:2, bound to a viral protein binding domain of SEQ ID NO:6 through a chemical linker of SEQ ID NO:3. In one embodiment, the peptide includes a dimerizing domain of SEQ ID NO:2, bound to a viral protein binding domain of SEQ ID NO:7 through a chemical linker of SEQ ID NO:3. In one embodiment, the peptide includes a dimerizing domain of SEQ ID NO:2, bound to a viral protein binding domain of SEQ ID NO:8 through a chemical linker of SEQ ID NO:3. In one embodiment, the peptide includes the sequence of SEQ ID NO:3. In one embodiment, the peptide is the sequence of SEQ ID NO:5.

In embodiments, the peptide is a first peptide including a first dimerizing domain, a first viral protein binding domain and a first chemical linker. In one embodiment, the first dimerizing domain has the sequence of SEQ ID NO:2, the first viral protein binding domain has the sequence of SEQ ID NO:1 and the first chemical linker has the sequence of SEQ ID NO:3. In one embodiment, the first dimerizing domain has the sequence of SEQ ID NO:2, the first viral protein binding domain has the sequence of SEQ ID NO:6 and the first chemical linker has the sequence of SEQ ID NO:3. In one embodiment, the first dimerizing domain has the sequence of SEQ ID NO:2, the first viral protein binding domain has the sequence of SEQ ID NO:7 and the first chemical linker has the sequence of SEQ ID NO:3. In one embodiment, the first dimerizing domain has the sequence of SEQ ID NO:2, the first viral protein binding domain has the sequence of SEQ ID NO:8 and the first chemical linker has the sequence of SEQ ID NO:3. In one embodiment, the first peptide includes the sequence of SEQ ID NO:5. In one embodiment, the first peptide is the sequence of SEQ ID NO:5.

In embodiments, the peptide is a second peptide including a second dimerizing domain, a second viral protein binding domain and a second chemical linker. In one embodiment, the second dimerizing domain has the sequence of SEQ ID NO:2, the second viral protein binding domain has the sequence of SEQ ID NO:1 and the second chemical linker has the sequence of SEQ ID NO:3. In one embodiment, the second dimerizing domain has the sequence of SEQ ID NO:2, the second viral protein binding domain has the sequence of SEQ ID NO:6 and the second chemical linker has the sequence of SEQ ID NO:3. In one embodiment, the second dimerizing domain has the sequence of SEQ ID NO:2, the second viral protein binding domain has the sequence of SEQ ID NO:7 and the second chemical linker has the sequence of SEQ ID NO:3. In one embodiment, the second dimerizing domain has the sequence of SEQ ID NO:2, the second viral protein binding domain has the sequence of SEQ ID NO:8 and the second chemical linker has the sequence of SEQ ID NO:3. In one embodiment, the second peptide includes the sequence of SEQ ID NO:5. In one embodiment, the second peptide is the sequence of SEQ ID NO:5.

In an aspect is provided an isolated nucleic acid encoding a peptide as disclosed herein, including embodiments thereof.

In an aspect is provided an expression vector including a nucleic acid as disclosed herein, including embodiments thereof.

In embodiments, the expression vector is a viral vector.

In an aspect is provided a method of treating a viral disease in a subject in need thereof, the method including administering to a subject a therapeutically effective amount of a peptide as disclosed herein including embodiment thereof, thereby treating an infectious disease in the subject. In embodiments, the viral disease is SARS. In embodiments, the viral disease is COVID-19.

In an aspect is provided a pharmaceutical composition including a therapeutically effective amount of a peptide as disclosed herein including embodiments thereof and a pharmaceutically acceptable excipient.

### EXAMPLES

### Example 1: Structural Analysis

ACE2 is the primary receptor used by the trimeric SARS-CoV2 spike protein to gain entry into the cell. Presumably, viral entry can be halted by blocking this interaction and significant effort and resources have been spent to identify and develop monoclonal antibodies to bind to the receptor binding domain (RBD) of the spike protein. Another approach is to use soluble ACE2. The issue with this approach is that the affinities are not high enough to efficiently inhibit viral entry; as indirect proof, ACE2 was shown to be shed in healthy tissues and thus there is a pool of ACE2 in the body [1]. However, as the spike protein is trimeric, it is possible to create multivalent ligands to enhance the affinity through avidity.

One possible approach is to fuse ACE2 to the IgG Fc domain, thus effectively replacing the Fab, to create a bivalent molecule. In order to enhance the affinity, both ACE2 receptors of the bivalent ACE2-Fc construct must be able to simultaneously bind to two RBD domains. This stipulation sets geometric constraints on the ACE2-Fc. Through numerous superpositions using available cryogenic electron microscopy (cryo-EM) structures of the trimeric spike protein, it became clear that the peptide linker between the ACE2 and Fc would have be extraordinarily long as the C-termini of the ACE2 were point away from each other and distant (over 200 Angstrom (Å)). Crude modeling indicated that the peptide linker would need to be about 40 residues (e.g., ACE2-40 residues-Fc). The same superpositions, however, showed the N-termini of the superimposed ACE2 receptors were very close. Applicants hereby provide the geometry and construction of bivalent and multivalent ACE2 decoys to block viral entry, and methods of using the same in treating COVID19.

To take advantage of this observation, we proposed to flip the order of the fusion—placing the Fc first. The resulting molecule would be Fc-11 residues-ACE2. The molecule has been made, purified and now is be characterized using in vitro neutralization assays. We are also testing the binding affinity using Surface Plasmon Resonance (SPR) and planning on obtaining a cryoEM structure of the Fc-ACE2 construct.

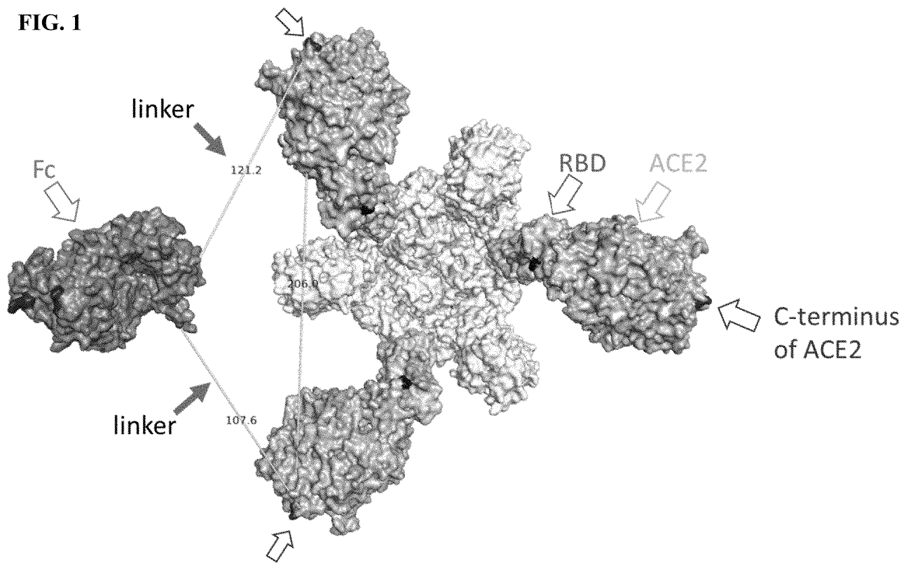

Many cytoplasmic domains are fused to the N-terminus of a crystallisable fragment (Fc) of an antibody as such Fc usually appears to improve expression of the construct and provides a convenient purification handle. Crystallisable fragments have also been used to make therapeutics such as etanercept and abatacept. The dimeric nature of the Fc can also improve the overall affinity due to avidity, or energy additivity. To realize gains in energy additivity, the geometry of the fused proteins such that they can engage with their partners simultaneously is critical. To that end, several investigators have generated ACE2-Fc fusions as a potential therapeutic to treat COVID19. As shown on FIG. 1, the trouble with this approach is the position of the C-terminus of ACE2 (red patches on lilac surface) point outward based on the superposition of these ACE2/RBD (wheat surface) structures on the trimeric spike protein (white surface). To fuse the ACE2 C-terminus to the N-terminus of an Fc (teal) requires an extraordinary long linker. The end-to-end distance between the N-termini is about 110 Å (Angstrom). The length of an amino acid is 3.54 Å. Thus a minimal linker would need to be 31 residues. To account for chemical and steric constraints, its likely at least another 4 residues on each end is necessary, affording a 39, or more, residue linker. Full avidity would be very difficult to achieve given the distance and entropic costs. To the best of our knowledge, the linker average 10 residues, which is way too short to bind to two RBDs intramolecularly.

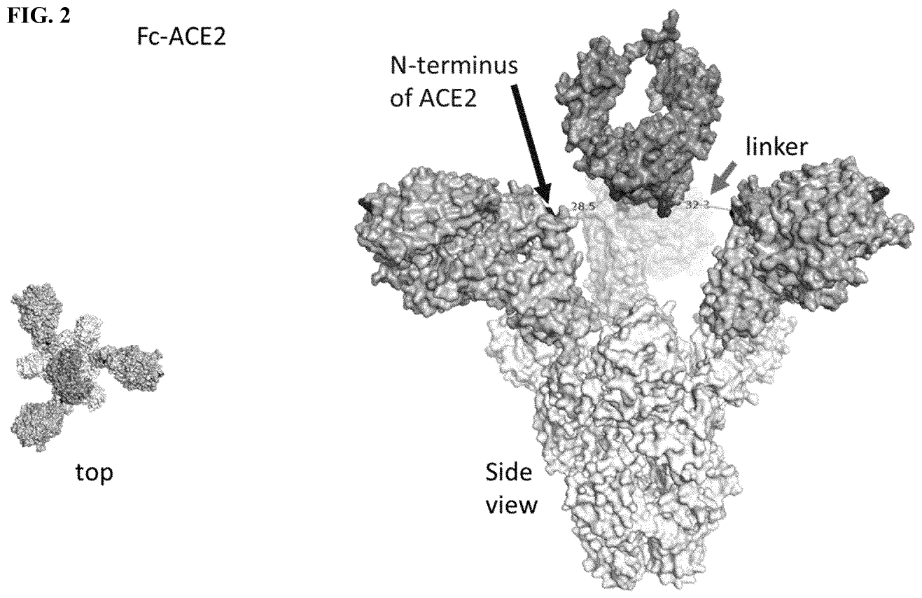

An alternative approach is to reverse the order by fusing the ACE2 to the C-terminus of the Fc. The reason for this is that the same superposition shows that the N-termini of the super imposed ACE2 are in close proximity, such that they point to each other's as opposed to pointing away from each other like the ACE2 C-termini. As shown in FIG. 2, the distance between the N-terminus of the super imposed ACE2 and the C-terminus of the Fc is much shorter, about 30 Å, or about 9 residues. Moreover, the steric interactions, are more favorable, and likely not requiring additional residues to alleviate steric restraints.

### Example 2: Results

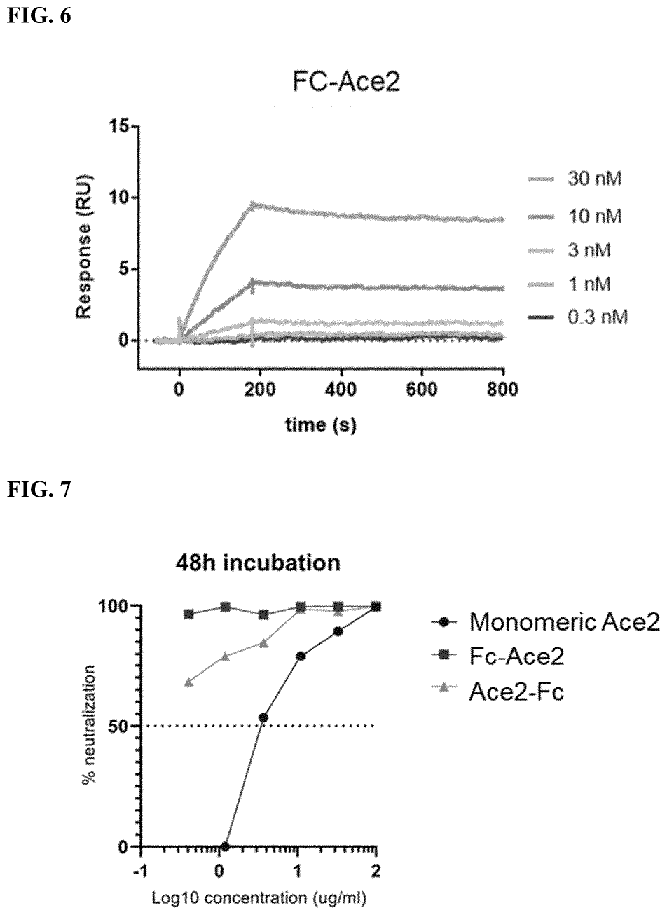

We observed that Fc-ACE2 was expressed at high levels (>120 mg/L transiently), and that it showed the same stability as ACE2-Fc constructs (FIG. 5).

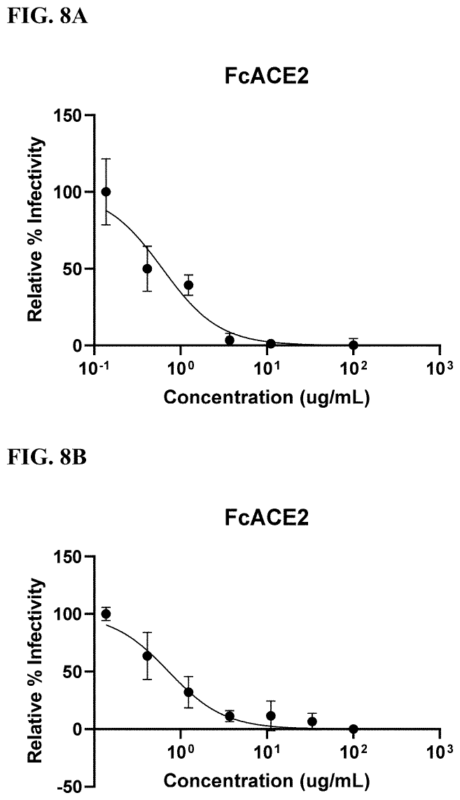

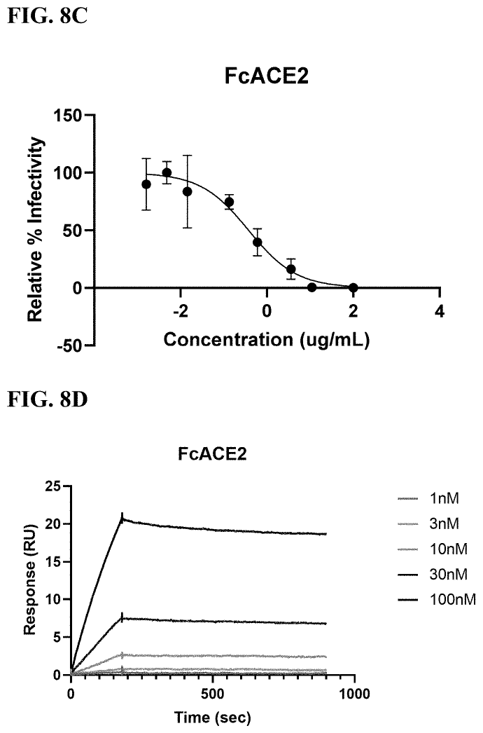

Fc-ACE2 was also observed to bind the trimeric spike with high affinity by Surface Plasmon Resonance at values beyond the instrument limits (FIG. 6) and to block viral entry using a pseudoviral assay (FIG. 7).

The Fc-ACE2 construct will be purified using readily available techniques such as ion exchange chromatography or protein A purification. The Fc-ACE2 construct will be useful against future SARS-CoV2 outbreaks, and will be less chance immunogenic once administered to a patient.

### REFERENCES

1. Jia H P, Look D C, Tan P, et al. Ectodomain shedding of angiotensin converting enzyme 2 in human airway epithelia. Am J Physiol Lung Cell Mol Physiol. 2009; 297(1):L84-L96. doi:10.1152/ajplung.00071.2009

INFORMAL SEQUENCE LISTING:

Viral protein binding domain-ACE2 domain (SEQ ID NO: 1)
STIEEQAKTFLDKFNHEAEDLFYQSSLASWNYNTNITEENVQNMNNAGDKWSAFLKEQSTLAQM
YPLQEIQNLTVKLQLQALQQNGSSVLSEDKSKRLNTILNTMSTIYSTGKVCNPDNPQECLLLEP
GLNEIMANSLDYNERLWAWESWRSEVGKQLRPLYEEYVVLKNEMARANHYEDYGDYWRGDYEVN
GVDGYDYSRGQLIEDVEHTFEEIKPLYEHLHAYVRAKLMNAYPSYISPIGCLPAHLLGDMWGRF
WTNLYSLTVPFGQKPNIDVTDAMVDQAWDAQRIFKEAEKFFVSVGLPNMTQGFWENSMLTDPGN
VQKAVCHPTAWDLGKGDFRILMCTKVTMDDFLTAHHEMGHIQYDMAYAAQPFLLRNGANEGFHE
AVGEIMSLSAATPKHLKSIGLLSPDFQEDNETEINFLLKQALTIVGTLPFTYMLEKWRWMVFKG
EIPKDQWMKKWWEMKREIVGVVEPVPHDETYCDPASLFHVSNDYSFIRYYTRTLYQFQFQEALC
QAAKHEGPLHKCDISNSTEAGQKLFNMLRLGKSEPWTLALENVVGAKNMNVRPLLNYFEPLFTW
LKDQNKNSFVGWSTDWSPYA
Dimerizing domain-Fc dimerizing domain (SEQ ID NO: 2)
DKTHTCPPCPAPELLGGPSVFLFPPKPKDTLMISRTPEVTCVVVDVSHEDPEVKFNWYVDGVEV
HNAKTKPREEQYASTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTV
DKSRWQQGNVFSCSVMHEALHNHYTQKSLSLSPGK
Peptide linker (SEQ ID NO: 3)
GGSASSGGGS
Full length IgG Fc (SEQ ID NO: 4)
QVQLVQSGGGVVQPGRSLRLSCASGFTFSRYTIHWVRQAPGKGLEWVAVMSYNGNNKHYADSV
NGRFTISRNDSKNTLYLNMNSLRPEDTAVYYCARIRDTAMFFAHWGQGTLVTVSSASTKGPSVF
PLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNHYTQKSLSLSPG
K
Fc-ACE2 full length construct (SEQ ID NO: 5)
DKTHTCPPCPAPELLGGPSVFLFPPKPKDTLMISRTPEVTCVVVDVSHEDPEVKFNWYVDGVEV
HNAKTKPREEQYASTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTV
DKSRWQQGNVFSCSVMHEALHNHYTQKSLSLSPGKGGSASSGGGSSTIEEQAKTFLDKFNHEAE
DLFYQSSLASWNYNTNITEENVQNMNNAGDKWSAFLKEQSTLAQMYPLQEIQNLTVKLQLQALQ
QNGSSVLSEDKSKRLNTILNTMSTIYSTGKVCNPDNPQECLLLEPGLNEIMANSLDYNERLWAW
ESWRSEVGKQLRPLYEEYVVLKNEMARANHYEDYGDYWRGDYEVNGVDGYDYSRGQLIEDVEHT
FEEIKPLYEHLHAYVRAKLMNAYPSYISPIGCLPAHLLGDMWGRFWTNLYSLTVPFGQKPNIDV
TDAMVDQAWDAQRIFKEAEKFFVSVGLPNMTQGFWENSMLTDPGNVQKAVCHPTAWDLGKGDFR
ILMCTKVTMDDFLTAHHEMGHIQYDMAYAAQPFLLRNGANEGFHEAVGEIMSLSAATPKHLKSI
GLLSPDFQEDNETEINFLLKQALTIVGTLPFTYMLEKWRWMVFKGEIPKDQWMKKWWEMKREIV
GVVEPVPHDETYCDPASLFHVSNDYSFIRYYTRTLYQFQFQEALCQAAKHEGPLHKCDISNSTE
AGQKLFNMLRLGKSEPWTLALENVVGAKNMNVRPLLNYFEPLFTWLKDQNKNSFVGWSTDWSPY
A
Enzymatically inactive ACE2 domain (Viral protein binding domain of
SEQ ID NO: 1 domain with ″R273S″ mutation) (SEQ ID NO: 6)
STIEEQAKTFLDKFNHEAEDLFYQSSLASWNYNTNITEENVQNMNNAGDKWSAFLKEQSTLAQM
YPLQEIQNLTVKLQLQALQQNGSSVLSEDKSKRLNTILNTMSTIYSTGKVCNPDNPQECLLLEP
GLNEIMANSLDYNERLWAWESWRSEVGKQLRPLYEEYVVLKNEMARANHYEDYGDYWRGDYEVN
GVDGYDYSRGQLIEDVEHTFEEIKPLYEHLHAYVRAKLMNAYPSYISPIGCLPAHLLGDMWGSF
WTNLYSLTVPFGQKPNIDVTDAMVDQAWDAQRIFKEAEKFFVSVGLPNMTQGFWENSMLTDPGN
VQKAVCHPTAWDLGKGDFRILMCTKVTMDDFLTAHHEMGHIQYDMAYAAQPFLLRNGANEGFHE
AVGEIMSLSAATPKHLKSIGLLSPDFQEDNETEINFLLKQALTIVGTLPFTYMLEKWRWMVFKG
EIPKDQWMKKWWEMKREIVGVVEPVPHDETYCDPASLFHVSNDYSFIRYYTRTLYQFQFQEALC
QAAKHEGPLHKCDISNSTEAGQKLFNMLRLGKSEPWTLALENVVGAKNMNVRPLLNYFEPLFTW
LKDQNKNSFVGWSTDWSPYA
Enzymatically inactive ACE2 domain (Viral protein binding domain of
SEQ ID NO: 1 domain with ″H345F″ mutation) (SEQ ID NO: 7)
STIEEQAKTFLDKFNHEAEDLFYQSSLASWNYNTNITEENVQNMNNAGDKWSAFLKEQSTLAQM
YPLQEIQNLTVKLQLQALQQNGSSVLSEDKSKRLNTILNTMSTIYSTGKVCNPDNPQECLLLEP
GLNEIMANSLDYNERLWAWESWRSEVGKQLRPLYEEYVVLKNEMARANHYEDYGDYWRGDYEVN
GVDGYDYSRGQLIEDVEHTFEEIKPLYEHLHAYVRAKLMNAYPSYISPIGCLPAHLLGDMWGRF
WTNLYSLTVPFGQKPNIDVTDAMVDQAWDAQRIFKEAEKFFVSVGLPNMTQGFWENSMLTDPGN
VQKAVCFPTAWDLGKGDFRILMCTKVTMDDFLTAHHEMGHIQYDMAYAAQPFLLRNGANEGFHE
AVGEIMSLSAATPKHLKSIGLLSPDFQEDNETEINFLLKQALTIVGTLPFTYMLEKWRWMVFKG
EIPKDQWMKKWWEMKREIVGVVEPVPHDETYCDPASLFHVSNDYSFIRYYTRTLYQFQFQEALC
QAAKHEGPLHKCDISNSTEAGQKLFNMLRLGKSEPWTLALENVVGAKNMNVRPLLNYFEPLFTW
LKDQNKNSFVGWSTDWSPYA
Enzymatically inactive ACE2 domain (Viral protein binding domain of
SEQ ID NO: 1 domain with ″H345S″ mutation) (SEQ ID NO: 8)
STIEEQAKTFLDKFNHEAEDLFYQSSLASWNYNTNITEENVQNMNNAGDKWSAFLKEQSTLAQM
YPLQEIQNLTVKLQLQALQQNGSSVLSEDKSKRLNTILNTMSTIYSTGKVCNPDNPQECLLLEP
GLNEIMANSLDYNERLWAWESWRSEVGKQLRPLYEEYVVLKNEMARANHYEDYGDYWRGDYEVN
GVDGYDYSRGQLIEDVEHTFEEIKPLYEHLHAYVRAKLMNAYPSYISPIGCLPAHLLGDMWGRF
WTNLYSLTVPFGQKPNIDVTDAMVDQAWDAQRIFKEAEKFFVSVGLPNMTQGFWENSMLTDPGN
VQKAVCSPTAWDLGKGDFRILMCTKVTMDDFLTAHHEMGHIQYDMAYAAQPFLLRNGANEGFHE
AVGEIMSLSAATPKHLKSIGLLSPDFQEDNETEINFLLKQALTIVGTLPFTYMLEKWRWMVFKG
EIPKDQWMKKWWEMKREIVGVVEPVPHDETYCDPASLFHVSNDYSFIRYYTRTLYQFQFQEALC
QAAKHEGPLHKCDISNSTEAGQKLFNMLRLGKSEPWTLALENVVGAKNMNVRPLLNYFEPLFTW
LKDQNKNSFVGWSTDWSPYA

1. A peptide comprising a dimerizing domain bound to a viral protein binding domain through a chemical linker, wherein said viral protein binding domain is bound to the C-terminus of said dimerizing domain.

2. The peptide of claim 1, wherein said viral protein binding domain is a Severe Acute Respiratory Syndrome (SARS)-coronavirus (CoV) protein binding domain or a Middle East Respiratory Syndrome (MERS)-coronavirus (CoV) protein binding domain.

3. (canceled)

4. The peptide of claim 1, wherein said viral protein binding domain is a spike protein binding domain.

5. (canceled)

6. (canceled)

7. The peptide of claim 1, wherein said viral protein binding domain is an angiotensin converting enzyme 2 (ACE2) domain.

8. The peptide of claim 1, wherein said viral protein binding domain comprises the sequence of SEQ ID NO:1, SEQ ID NO:6, SEQ ID NO:7 or SEQ ID NO:8.

9. The peptide of claim 1, wherein said dimerizing domain is a Fc dimerizing domain.

10. The peptide of claim 1, wherein said dimerizing domain comprises a CH2 and a CH3 domain.

11.-15. (canceled)

16. The peptide of claim 1, wherein said chemical linker is a peptide linker.

17. (canceled)

18. The peptide of claim 16, wherein said peptide linker has a length of less than 20 amino acid residues.

19.-25. (canceled)

26. The peptide of claim 1, wherein said peptide is a first peptide and said dimerizing domain is a first dimerizing domain.

27. The peptide of claim 26, wherein said first dimerizing domain is bound to a second peptide comprising:
a second dimerizing domain bound to a second viral protein binding domain through a second chemical linker, wherein said first viral protein binding domain is bound to the C-terminus of said second dimerizing domain; and
wherein said first dimerizing domain and said second dimerizing domain are covalently bound together thereby binding said first peptide to said second peptide.

28. The peptide of claim 27, wherein said second viral protein binding domain is a Severe Acute Respiratory Syndrome (SARS)-coronavirus (CoV) protein binding domain or a Middle East Respiratory Syndrome (MERS)-coronavirus (CoV) protein binding domain.

29. (canceled)

30. The peptide of claim 27, wherein said second viral protein binding domain is a spike protein binding domain.

31.-34. (canceled)

35. The peptide of claim 27, wherein said second dimerizing domain is an Fe dimerizing domain.

36.-53. (canceled)

54. A peptide complex comprising:
(i) a first peptide comprising a first dimerizing domain bound to a first viral protein binding domain through a first chemical linker, wherein said first viral protein binding domain is bound to the C-terminus of said first dimerizing domain; and
(ii) a second peptide comprising a second dimerizing domain bound to a second viral protein binding domain through a second chemical linker, wherein said second viral protein binding domain is bound to the C-terminus of said second dimerizing domain;
wherein said first dimerizing domain and said second dimerizing domain are bound together thereby binding said first peptide to said second peptide.

55. An isolated nucleic acid encoding a peptide of claim 1.

56. An expression vector comprising the nucleic acid of claim 55.

57. (canceled)

58. A method of treating a viral disease in a subject in need thereof, said method comprising administering to a subject a therapeutically effective amount of a peptide of claim 1, thereby treating an infectious disease in said subject.

59. The method of claim 58, wherein said viral disease is SARS.

60. (canceled)

61. A pharmaceutical composition comprising a therapeutically effective amount of a peptide of claim 1 and a pharmaceutically acceptable excipient.


SEQUENCE LISTING (8 sequences)

SEQ ID NO: 1 - protein, 596 aa, Artificial Sequence
  Viral protein binding domain - ACE2 domain
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 2 - protein, 227 aa, Artificial Sequence
  Dimerizing domain - Fc dimerizing domain
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 10 aa, Artificial Sequence
  Peptide linker
GGSASSGGGS

SEQ ID NO: 4 - protein, 449 aa, Artificial Sequence
  Full length IgG Fc
QVQLVQSGGGVVQPGRSLRLSCAASGFTFSRYTIHWVRQAPGKGLEWVAVMSYNGNNKHYADSVNGRFTISRNDSKNTLYLNMNSLRPEDTAVYYCARIRDTAMFFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 833 aa, Artificial Sequence
  Fc-ACE2 full length construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSASSGGGSSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 6 - protein, 596 aa, Artificial Sequence; identical to SEQ ID NO: 1
  Enzymatically inactive ACE2 domain (Viral protein binding domain of ACE2 domain with “R273S” mutation)

SEQ ID NO: 7 - protein, 596 aa, Artificial Sequence
  Enzymatically inactive ACE2 domain (Viral protein binding domain of ACE2 domain  with “H345F” mutation)
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCFPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 8 - protein, 596 aa, Artificial Sequence
  Enzymatically inactive ACE2 domain (Viral protein binding domain of ACE2 domain  with “H345S” mutation)
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCSPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA